(19) United States
(12) Patent Application Publication
(10) Pub. No.: US 2015/0322398 A1
(43) Pub. Date: Nov. 12, 2015

(54) BIOMASS PROCESSING DEVICE AND PROCESSING METHOD

(71) Applicant: TSUKISHIMA KIKAI CO., LTD., Tokyo (JP)

(72) Inventors: Tomoki HAYAKAWA, Tokyo (JP); Hideaki MIZUNO, Tokyo (JP)

(21) Appl. No.: 14/409,404

(22) PCT Filed: Mar. 27, 2013
(86) PCT No.: PCT/JP2013/058944

§ 371 Date: Dec. 18, 2014

(30) Foreign Application Priority Data
Jun. 21, 2012 (JP) 2012-140019

(51) Int. Cl. C12M 1/00; C12M 1/34; C12P 19/14; C12P 7/10; C12P 7/56; C12P 7/46; C12M 1/12; C12P 19/02

(52) U.S. Cl. CPC C12M 23/58; C12M 29/18; C12M 37/00; C12M 41/44; C12P 19/02; C12P 7/10; C12P 7/56; C12P 7/46; C12P 19/14; C12P 2201/00; C12P 2203/00

(57) ABSTRACT

A biomass treatment device and a biomass treatment method, in which contamination is prevented and a final product can be highly concentrated. The biomass treatment device includes a pretreatment device for pretreating a biomass, a slurrying device for slurrying the pretreated biomass, and a saccharification device for saccharifying the slurried biomass with a saccharifying enzyme. A return line for returning a part of an internal fluid in the saccharification device to the slurrying device is provided, and the pretreated biomass from the pretreatment device and the internal fluid returned through the return line are mixed in the slurrying device so as to generate biomass slurry.

### TECHNICAL FIELD

The present invention relates to a biomass treatment device including a saccharification device for hydrolyzing a fibrous material such as cellulose, which is a main component of biomass, to saccharides by a saccharifying enzyme, and a pretreatment device for performing a pretreatment before a saccharification reaction with a saccharifying enzyme; and a method for treating biomass using the same.

### BACKGROUND ART

The biomass containing cellulose and hemicellulose of bagasse, wheat straw, rice straw, palm residue, paper, and the like has been mainly used as animal feed, and as recent advances in a cellulose saccharification technique, an application where the biomass is used as a raw material of ethanol or an organic acid is gradually being developed. In producing the ethanol or the organic acid from the biomass containing the cellulose and hemicellulose, a fibrous material such as the cellulose and hemicellulose, which is a main component of the biomass, is subjected to a pretreatment such as an acid treatment, a hydrothermal treatment and the like; the pretreated biomass is subjected to sterilization; the sterilized biomass is reacted with a saccharifying enzyme to obtain a saccharified solution containing saccharides such as glucose, xylose, arabinose, galactose, and mannose; and the saccharides are converted to the ethanol and the like by a fermentation method using a microorganism such as yeast. In addition, is also frequently used a technique in which enzymatic saccharification and fermentation are performed at the same time in the same tank for the biomass after the pretreatment and the sterilization (simultaneous saccharification and fermentation). Further, instead of performing the sterilization after the pretreatment, the sterilization may be performed at the same time as the pretreatment. The sterilization is performed in order to prevent contamination, because due to such contamination, the produced amount of the saccharides or that of the fermentation product (in simultaneous saccharification and fermentation) would be decreased while impurities would be produced.

In producing ethanol and the like from the biomass, it is important to increase the concentration of the saccharides or that of the fermentation product in order to reduce the load of purification in a final step. The high concentration of the saccharides or that of the fermentation product can be achieved by preventing the above contamination as well as by increasing the concentration of the raw material in a saccharification device.

For highly concentrating the saccharides or the fermentation product, there is a method in which pretreated biomass at a high concentration is fed to a saccharification device (for example, a bioreactor), and the pretreated biomass is saccharified at the high concentration in the bioreactor. The pretreated biomass at the high concentration appears like a solid state so that fluidity is very low in the bioreactor, and therefore, a high power mixing device such as a rotating drum, and a rotary blade is required (Patent Literature 1). However, even by such a high power mixing device, sufficient mixing cannot be attained, therefore, there is a problem that conversion of the pretreated biomass to the saccharides cannot be increased.

In addition, since the pretreated biomass at the high concentration appears like a solid state, a belt conveyer, a flight conveyer, a screw conveyer, a bucket conveyer, or the like is applied for conveying the pretreated biomass from the pretreatment device to the bioreactor. However, there is a problem that the pretreated biomass in general easily sticks to the solid conveying unit, such as the conveyer, which causes difficulty in washing, resulting in contamination. Further, there is another problem that since plural bioreactors are usually used, plural belt conveyers and the like are also required for connecting the pretreatment device and the bioreactors, resulting in high construction cost.

On the other hand, there is a fed-batch method by which the high concentration of produced saccharides can be achieved and a sufficient mixing property is ensured without a high power mixing device. The fed-batch method is a technique for operating a bioreactor in which the concentration of biomass is set so as to ensure sufficient fluidity, and additional biomass is fed in any time by the amount which offsets the amount dissolved in saccahrification. However, such a fed-batch method still has a problem of contamination, because the high concentration of pretreated biomass in a solid state has to be conveyed from a pretreatment device to the bioreactor via a conveyor or the like.

On the other hand, in order to prevent the contamination, as a method for conveying pretreated biomass without a solid conveying unit such as a conveyer, there is a technique in which a liquid is mixed to the pretreated biomass so as to be slurried, and the biomass slurry is pump-transported to a bioreactor through a pipe. The technique has an advantage of low construction cost due to the absence of a conveyer or the like, and further another advantage of low contamination due to good washability. However, the technique has a problem of an insufficient ability of increasing the concentration of saccharides, because the concentration of the pretreated biomass in the bioreactor cannot be set over a concentration that enables slurry transportation.

### CITATION LIST

### Patent Literature

### Patent Literature 1: Japanese Patent Application National Publication No. 2008-521396

### SUMMARY OF INVENTION

### Technical Problem

The main object of the present invention is to provide a biomass treatment device and a biomass treatment method by which contamination is prevented as well as a final product can be highly concentrated.

### Solution to Problem

The present invention to solve the above problems is as follows.

### <Invention According to Claim 1>

A biomass treatment device, including:

a pretreatment device for pretreating a biomass;

a slurrying device for slurrying the pretreated biomass; and

a saccharification device for saccharifying the slurried biomass with a saccharifying enzyme,

the biomass treatment device comprising:

a return line for returning a part of an internal fluid in the saccharification device to the slurrying device; and

the slurrying device where the pretreated biomass from the pretreatment device and the internal fluid returned through the return line are mixed so as to generate a biomass slurry.

### Operation and Effect

In the present invention, the part of the internal fluid in the saccharification device is returned to the slurrying device through the return line, and the returned internal fluid and the pretreated biomass from the pretreatment device are mixed in the slurrying device to generate the biomass slurry. Accordingly, it is unnecessary to feed additional water and the like for the slurrying so that the amount of the internal fluid in the saccharification device can be set as low as possible and the total amount of the biomass to be fed into the saccharification device can be increased.

### <Invention According to Claim 2>

The biomass treatment device according to claim 1,

wherein the saccharification device is a simultaneous saccharification and fermentation device where a saccharification step for saccharifying the slurried biomass with the saccharifying enzyme and a fermentation step for fermenting the saccharified biomass with a fermentive microorganism are performed simultaneously and

a part of an internal fluid in the simultaneous saccharification and fermentation device is returned to the slurrying device.

### Operation and Effect

Since the saccharified biomass can be converted promptly to fermentation product in the simultaneous saccharification and fermentation device, accumulation of the saccharified biomass as an intermediate product can be prevented so as to prevent inactivation of the saccharifying enzyme.

### <Invention According to Claim 3>

The biomass treatment device according to claim 1 or 2, wherein a cooler is provided in the return line so as to cool the internal fluid returned therethrough.

### Operation and Effect

Since the internal fluid returning through the return line is cooled by the cooler, the pretreated biomass can be sufficiently cooled by the returned internal fluid in the slurrying device. As a result, when the internal fluid is fed into the slurrying device, or when the biomass slurry is fed into the saccharification device or the simultaneous saccharification and fermentation device, it is possible to prevent inactivation of the saccharifying enzyme and that of the fermentive microorganism.

### <Invention According to Claim 4>

The biomass treatment device according to any one of claims 1 to 3, wherein a cooler is provided in a feed line, which is provided for feeding the biomass slurry from the slurrying device to the saccharification device, so as to cool the biomass slurry.

### Operation and Effect

Since the biomass slurry passing through the feed line is cooled by the cooler, the temperature of the biomass slurry can be lowered. As a result, when the biomass slurry is fed into the saccharification device, it is possible to prevent inactivation of the saccharifying enzyme and that of the fermentive microorganism.

### <Invention According to Claim 5>

The biomass treatment device according to claim 1 or 2, wherein in the pretreatment device, at least one of a thermal treatment, a hydrothermal treatment, an acid treatment, or an alkali treatment is performed and sterilization of the biomass is performed through the treatment.

### Operation and Effect

Since sterilization is performed in the pretreatment device, it is unnecessary to perform further sterilization by a sterilizer provided in a step after the pretreatment.

### <Invention According to Claim 6>

The biomass treatment device according to claim 1 or 2, wherein a slurry transportation pipe is arranged between the slurrying device and the saccharification device so as to transform the biomass slurry from the slurrying device to the saccharification device through the slurry transportation pipe.

### Operation and Effect

Since the biomass slurry is, after the pretreatment, transported by using the transportation pipe, the contamination can be prevented, and further construction cost can be lowered.

### <Invention According to Claim 7>

The biomass treatment device according to claim 1 or 2, wherein a slurry concentration measuring unit for the internal fluid is provided in the return line or in the saccharification device, and if a slurry concentration of the internal fluid is high, an amount of the internal fluid to be returned is increased or an amount of the biomass to be fed to the slurrying device is decreased.

### Operation and Effect

When the slurry concentration of the internal fluid is high, the amount of the internal fluid to be returned should be increased or the amount of the biomass to be fed to the slurrying device should be decreased. By doing so, the slurry concentration can be decreased in the slurrying device and in the feed line so that fluidity can be improved and clogging can be prevented there.

### <Invention According to Claim 8>

The biomass treatment device according to claim 1, 2, 6 or 7, wherein a slurry concentration measuring unit for the biomass slurry is provided in a feed line, which is provided for feeding the biomass slurry from the slurrying device to the saccharification device, and if a slurry concentration of the biomass slurry is high, an amount of the internal fluid to be returned is increased or an amount of the biomass to be fed to the slurrying device is decreased.

### Operation and Effect

The same operation and effect as those by claim 7 can be exerted.

### <Invention According to Claim 9>

A method for treating a biomass, including:

a step for pretreating the biomass in a pretreatment device;

a step for slurrying the pretreated biomass in a slurrying device; and

a step for saccharifying the slurried biomass by reacting with a saccharifying enzyme in a saccharification device,

the method comprising

returning, through a returning line, a part of an internal fluid in the saccharification device to the slurrying device; and

slurrying the biomass in the slurrying device where the pretreated biomass from the pretreatment device and the internal fluid returned through the returning line are mixed.

### Operation and Effect

The same operation and effect as those by claim 1 can be exerted.

### <Invention According to Claim 10>

A method for treating a biomass, including:

a step for pretreating the biomass in a pretreatment device;

a step for slurrying the pretreated biomass in a slurrying device; and

a step for simultaneous saccharification and fermentation in a simultaneous saccharification and fermentation device where the slurried biomass is reacted with a saccharifying enzyme so as to be saccharified and at the same time the saccharified biomass is reacted with a fermentive microorganism so as to be fermented,

the method comprising

returning, through a returning line, a part of an internal fluid in the simultaneous saccharification and fermentation device to the slurrying device; and

slurrying the pretreated biomass in the slurrying device where the pretreated biomass from the pretreatment device and the internal fluid returned through the return line are mixed.

### Operation and Effect

The same operation and effect as those by claim 2 can be exerted.

### Advantageous Effects of Invention

According to the present invention, the contamination is prevented and additionally the final product can be highly concentrated.

### BRIEF DESCRIPTION OF DRAWINGS

FIG. 1 is the flow diagram of the biomass treatment according to the present invention.

FIG. 2 is the flow diagram of the biomass treatment according to the present invention showing a case where simultaneous saccharification and fermentation is performed.

### DESCRIPTION OF EMBODIMENTS

### [Biomass Treatment Step]

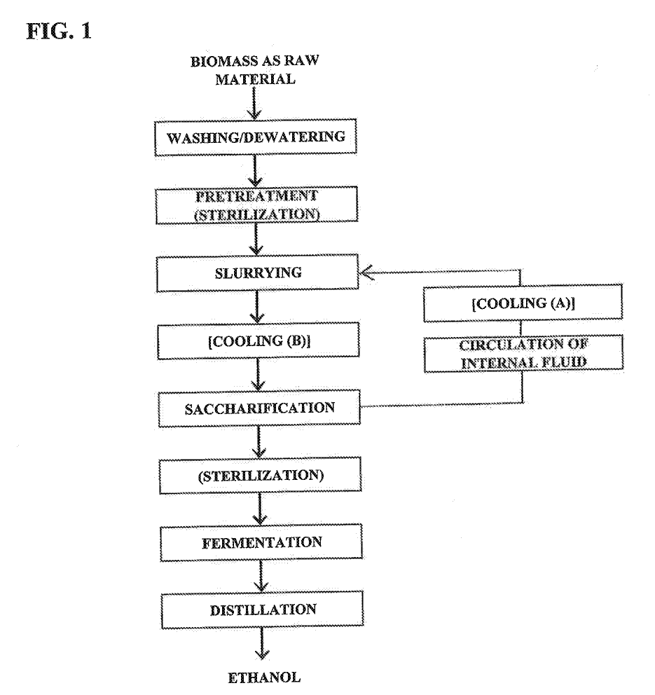

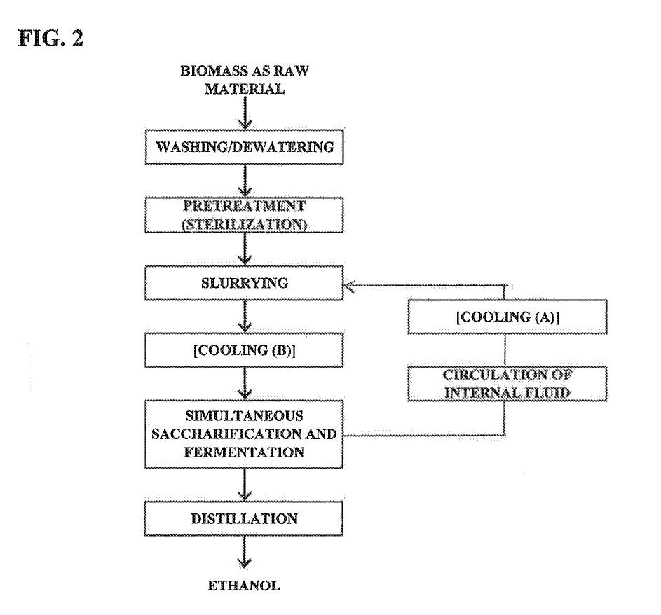

FIG. 1 and FIG. 2 show the schematic flow diagram of the biomass treatment.

A device according to the present invention can suitably be used for biomass containing cellulose and hemicellulose of bagasse, wheat straw, palm residue, corn stover, palm coconut residue, cassava residue, wood chips, wastewood, jute, kenaf, used paper, and the like.

### (Washing Step)

The biomass containing the cellulose and hemicellulose is separated from foreign materials such as sand and stones by using water in a washing step, and a large part of the water added in the washing step is removed in a dewatering step. On the other hand, the same effects can be obtained even by a method for separating the foreign materials without using water as in gravimetric separation with a pneumatic cyclone.

### (Pretreatment and Sterilization Step)

The biomass is a composite, in which mainly three components of cellulose, hemicellulose, and lignin are firmly bound, and further a cellulose molecule itself has a hard crystal structure, therefore, without any treatment, the cellulase as a saccharifying enzyme cannot approach to the cellulose molecule. It is required thus that the biomass after dewatering is pretreated by any of a thermal treatment, a hydrothermal treatment, an acid treatment, an alkali treatment, a milling, wood rotting fungi, and the like in the pretreatment device so as to loosen binding of the three components. By doing so, the cellulase as the saccharifying enzyme is easily brought into contact with the cellulose molecule, and the enzymatic saccharification is accelerated. After that, sterilization is generally performed in order to prevent contamination. However, when the pretreatment of any of the thermal treatment, the hydrothermal treatment (steaming, steam explosion), the acid treatment (a dilute acid process and a concentrated acid process using sulfuric acid, nitric acid, phosphoric acid, and the like, or a SO2 impregnated steam explosion process), and an alkali treatment (a caustic soda process, a sodium sulfite process, an ammonia process, or a calcium hydroxide process) is performed, the pretreatment has a sterilization effect. That is to say, this has an advantage that the sterilization is not required to be performed separately from the pretreatment step.

### (Slurrying Step)

If the pretreated biomass is conveyed to a saccharification device with a transportation unit such as a conveyer, the contamination would be possibly occurred as described above. Accordingly, in the present invention, before the saccharification, the pretreated biomass is slurried. Specifically, the pretreated biomass is transported to a slurrying device where the pretreated biomass and a liquid in the slurring device are mixed and the mixture is slurried such that the concentration of pretreated biomass is around 5% or less. The slurrying device may be an agitation vessel, or a static mixer, or may simply be a confluence point between a pipe through which the pretreated biomass transfers and a pipe through which a returned internal fluid flows. The slurrying device is connected to the saccharification device by a transportation pipe. This transportation pipe is used as a feed line. The biomass slurry is pump-transported to the saccharification device through this transportation pipe as the feed line so as to be subjected to a saccharification step. Alternatively, instead of the transportation pump, it is possible to transport the biomass slurry driven by pressure difference between the slurrying device and the saccharification device. As described above, since the biomass slurry can be transported through the pipe without using a conveyer and the like, washing can be carried out easily so that possibility of contamination can be reduced.

### (Saccharification Step)

The slurried biomass is saccharified in the saccharification device. A typical saccharification device is a bioreactor. In the saccharification device, the slurried biomass is decomposed mainly by the action of three kinds of cellulases (endoglucanase (EG), cellobiohydrolase (CBH), and β-glucosidase (BGL)). The cellulase is added in the amount of 3 to 30 FPU/g, and preferably 5 to 20 FPU/g of the dry weight of the biomass as a raw material (FPU/g is a unit of enzymatic activity of the cellulase which produces 10.8 mg of glucose on filter paper for 60 minutes). A simultaneous saccharification and fermentation device described below is also used as a saccharification device in the present invention.

### (Fermentation and Distillation Step)

The saccharified material is further converted to organic fuel such as ethanol, an organic acid by yeast or bacterium. In particular, ethanol production through fermentation and distillation is general case, which is used as a biofuel. In order to convert saccharides to the ethanol, yeast of Saccharomyces, Schizosaccharomyces, Kluyveromyces, Pichia, or Candida; a bacterium of Zymomonas, or Clostridium; or a genetically modified microorganism induced with a specific gene thereof can be used. The ethanol fermentative bacterium is inoculated, for example, in a case of Saccharomyces cerevisiae, in an amount of 1 to 100 g wet-weight/L, and preferably 5 to 50 g wet-weight/L-volume of fermentation broth. In addition, in order to convert the saccharides to lactic acid, for example, a filamentous fungus such as Rhizopus oryzae can be used. Further, in order to convert the saccharides to succinic acid, for example, a Coryneform bacterium, Bacillus, Rhizobium, or the like can be used.

### (Internal Fluid Returning Step)

The present invention is particularly characterized by a feature of returning an internal fluid in the saccharification device to the slurrying device. As described above, if a liquid, which does not contain the saccharides, is added into the slurrying device for slurrying the pretreated biomass, the concentration of the saccharides to be generated in the saccharification device cannot be sufficiently increased. Instead of the above liquid, which does not contain the saccharides, a liquid, which contains the saccharides, such as the internal fluid in the saccharification device, is used in the present invention. This means that the saccharides are contained also in the liquid used for the slurrying, thereby the saccharides concentration in the saccharification device can be increased.

Such returning of the internal fluid is performed by connecting the saccharification device to the slurrying device with the transportation pipe, through which the part of the internal fluid in the saccharification device is pump-transported to the slurrying device. Alternatively, instead of a transportation pump, it is possible to be driven by pressure difference between the slurrying device and the saccharification device.

The composition of the internal fluid to be returned is a biomass residue, e.g. lignin which has not been saccharified, the generated saccharides, the slight amount of the saccharifying enzyme, and the like in addition to the initially contained water in the saccharification device. Further, when the simultaneous saccharification and fermentation device explained below is used, the composition is in addition to the substances described above, the generated ethanol, the generated organic acid, fermentive microorganisms, and the like.

The amount of the internal fluid in the saccharification device, which is to be returned to the slurrying device, and the amount of the pretreated biomass, which is to be fed to the slurrying device, are determined by the following equation.

Given the amount of a solid in the pretreated biomass, which is to be fed from the pretreatment device to the slurrying device is 1 kg/h; the amount of a liquid in the pretreated biomass is z kg/h, the amount of a solid (composed mainly of lignin) contained in the internal fluid to be returned from the saccharification device to the slurrying device is x kg/h, the amount of the liquid in the slurry is y kg/h, and the slurry concentration m, which enables the slurry transportation from the slurrying device to the saccharification device, is 5%, the equation is shown below.

m=(1+x)/(1+x+y+z)×100≦5(%) [Mathematical 1]

That is, when the slurry concentration of the internal fluid to be returned, n (=x÷(x+y)×100) % is high, the amount of the internal fluid to be returned is required to be larger or the amount of the biomass fed to the slurrying device is required to be smaller than when the slurry concentration is low, so that the slurry concentration m becomes 5% or less. Therefore, it is preferable that a slurry concentration measuring unit for the internal fluid to be returned is provided in the return line or the saccharification device so that the amount of the internal fluid to be returned (x+y (kg/h)) is adjusted based on the measured slurry concentration. Alternatively, it is possible that the slurry concentration measuring unit is provided in the feed line for feeding the slurry from the slurrying device to the saccharification device so that the amount of the internal fluid to be returned (x+y (kg/h)) or the amount of the biomass fed to the slurrying device is adjusted based on the measured slurry concentration.

The slurry concentration m, which enables the transportation from the slurrying device to the saccharification device, varies depending on the shape of the solid substance contained in the slurry. More specifically, when the biomass is crushed before the pretreatment, a fibrous material is finely cut so that the fibrous material contained in the slurry tends to be short. Accordingly, fluidity is relatively well, which enables the transportation of the biomass slurry even if the slurry concentration m is around 8 to 10%. However, when the biomass is not crushed, the fibrous material contained in the slurry tends to be long and the fluidity of the slurry is lowered. In such situation, if the slurry concentration m is higher than 5%, the transportation of the biomass slurry becomes difficult.

### (Cooling Step)

Since the saccharification device is usually operated at 25 to 50° C., the temperature of the internal fluid to be returned through the return line is usually 25 to 50° C. On the other hand, since it is often that the pretreatment is performed in the presence of steam at the high temperature of 100° C. or more, the pretreated biomass generally has the temperature around 80° C. However, the saccharifying enzyme is generally inactivated just because the temperature exceeds 60° C. Therefore when the returned internal fluid is brought into contact with the pretreated biomass, the pretreated biomass has to be cooled quickly. In order to do so, it is preferable to provide a cooler (A) in the return line. By the cooler (A), the temperature of the internal fluid, which is being returned, is cooled to the temperature by around 2 to 12° C. Accordingly, the pretreated biomass can be cooled quickly upon the mixing of the returned internal fluid and the pretreated biomass, resulting in the small amount of the inactivated saccharifying enzyme. Further, by the mixture of the cooled internal fluid and the pretreated biomass, the temperature of the biomass slurry can be lowered to around the same temperature as that of the internal fluid in the saccharification device, and the saccharifying enzyme in the saccharification device can be prevented from the inactivation.

In addition, the cooler (B) may be provided in a feed line, which is provided for feeding the biomass slurry from the slurrying device to the saccharification device, in order to cool the biomass slurry before being fed to the saccharification device. As compared with the cooler (A) stated before, due to the cooler (B), since the temperature between the slurry tank and the cooler (B) is higher, the risk of inactivation of the enzyme is increased. However, by decreasing a distance between the slurry tank and the cooler (B), the operation can be performed in the practically acceptable inactivation range of the enzyme.

It is sufficient to provide only one of either the cooler (A) or the cooler (B), however, both may be provided in order to make assurance doubly sure. In a case where only one of either the cooler (A) or the cooler (B) is provided, it is desirable to provide the cooler (A) in order to prevent the inactivation of the saccharifying enzyme in a returning saccharified solution. However, since there is a disadvantage that the construction cost of the cooler (A) is higher than that of the cooler (B), the cooler (B) may be provided from the view point of the cost.

### (Simultaneous Saccharification and Fermentation Step)

FIG. 2 is the flow diagram of the treatment of the biomass according to the present invention, showing the simultaneous saccharification and fermentation step. In other words, FIG. 2 shows that saccharification and fermentation are performed at the same time in one device, although FIG. 1 shows that the saccharification and the fermentation are performed separately in different devices.

When lignocellulosic biomass is saccharified (hydrolyzed) to the glucose with the cellulase, there is a problem that the enzymatic activity is inhibited by the accumulation of the generated glucose, and the saccharification yield thereof is lowered. However, in the simultaneous saccharification and fermentation, the saccharification with a saccharifying enzyme and the ethanol fermentation of the saccharified material with yeast and the like can be performed at the same time. Accordingly, the enzymatic activity is rarely inhibited by the accumulation of the generated glucose, because before that, fermentative bacterium such as the yeast converts the saccharides, resulting in an advantage that the enzymatic activity can be maintained. Therefore, it is desirable to adopt the treatment flow shown in FIG. 2, comparing with that shown in FIG. 1.

In the simultaneous saccharification and fermentation in FIG. 2, the simultaneous saccharification and fermentation device and a slurrying device are connected by a transportation pipe, which is used as a return line. Part of the internal fluid in the simultaneous saccharification and fermentation device is transported to the slurrying device through the return line. The returning of the internal fluid is performed in the same way as in the saccharification device stated before, therefore, the detailed description is omitted herein.

Further, a cooler (A) and a cooler (B) are provided for cooling in the same way as in case of FIG. 1 except that inside of the saccharification and fermentation device should be maintained at a constant temperature by removing heat generated through the fermentation step, but through the saccharification step, heat is not generated. In cooling by the cooler (A) and the cooler (B), the temperature of biomass slurry to be supplied to the simultaneous saccharification and fermentation device is lowered than the temperature of the internal fluid in the simultaneous saccharification and fermentation device by around 0 to 5° C., and such biomass slurry is fed to the simultaneous saccharification and fermentation device so that the internal fluid in the simultaneous saccharification and fermentation device can be maintained at the constant temperature. In addition, when fermentive microorganisms present in the returning internal fluid and in the simultaneous saccharifying fermentation device are brought into contact with the biomass slurry, if it had a high temperature, inactivation of the fermentive microorganisms would be caused. However, cooling by the cooler (A) and the cooler (B) contributes to prevention of such inactivation of the fermentive microorganisms due to the biomass slurry which does not have high temperature. Detailed description, which explains steps other than the above steps performed by the simultaneous saccharification and fermentation device, is omitted, because it would be the same as the steps performed by the saccharification device shown in FIG. 1.

1. A biomass treatment device, including:
a pretreatment device for pretreating a biomass;
a slurrying device for slurrying the pretreated biomass; and
a saccharification device for saccharifying the slurried biomass with a saccharifying enzyme,
the biomass treatment device comprising:
a return line for returning a part of an internal fluid in the saccharification device to the slurrying device; and
the slurrying device where the pretreated biomass from the pretreatment device and the internal fluid returned through the return line are mixed so as to generate a biomass slurry.

2. The biomass treatment device according to claim 1,
wherein the saccharification device is a simultaneous saccharification and fermentation device where a saccharification step for saccharifying the slurried biomass with the saccharifying enzyme and a fermentation step for fermenting the saccharified biomass with a fermentive microorganism are performed simultaneously and
a part of the internal fluid in the simultaneous saccharification and fermentation device is returned to the slurrying device.

3. The biomass treatment device according to claim 1, wherein a cooler is provided in the return line so as to cool the internal fluid returned therethrough.

4. The biomass treatment device according to claim 1, wherein a cooler is provided in a feed line, which is provided for feeding the biomass slurry from the slurrying device to the saccharification device, so as to cool the biomass slurry.

5. The biomass treatment device according to claim 1, wherein in the pretreatment device, at least one of a thermal treatment, a hydrothermal treatment, an acid treatment, or an alkali treatment is performed and sterilization of the biomass is performed through the treatment.

6. The biomass treatment device according to claim 1, wherein a slurry transportation pipe is arranged between the slurrying device and the saccharification device so as to transfer the biomass slurry from the slurrying device to the saccharification device through the slurry transportation pipe.

7. The biomass treatment device according to claim 1, wherein a slurry concentration measuring unit for the internal fluid is provided in the return line or in the saccharification device, and if a slurry concentration of the internal fluid is high, an amount of the internal fluid to be returned is increased or an amount of the biomass to be fed to the slurrying device is decreased.

8. The biomass treatment device according to claim 1, wherein a slurry concentration measuring unit for the biomass slurry is provided in a feed line, which is provided for feeding the biomass slurry from the slurrying device to the saccharification device, and if a slurry concentration of the biomass slurry is high, an amount of the internal fluid to be returned is increased or an amount of the biomass to be fed to the slurrying device is decreased.

9. A method for treating a biomass, including:
a step for pretreating the biomass in a pretreatment device;
a step for slurrying the pretreated biomass in a slurrying device; and
a step for saccharifying the slurried biomass by reacting with a saccharifying enzyme in a saccharification device,
the method comprising
returning, through a returning line, a part of an internal fluid in the saccharification device to the slurrying device; and
slurrying the biomass in the slurrying device where the pretreated biomass from the pretreatment device and the internal fluid returned through the returning line are mixed.

10. A method for treating a biomass, including:
a step for pretreating the biomass in a pretreatment device;
a step for slurrying the pretreated biomass in a slurrying device; and
a step for simultaneous saccharification and fermentation in a simultaneous saccharification and fermentation device where the slurried biomass is reacted with a saccharifying enzyme so as to be saccharified and at the same time the saccharified biomass is reacted with a fermentive microorganism so as to be fermented,
the method comprising
returning, through a returning line, a part of an internal fluid in the simultaneous saccharification and fermentation device to the slurrying device; and
slurrying the pretreated biomass in the slurrying device where the pretreated biomass from the pretreatment device and the internal fluid returned through the return line are mixed.
